(19) United States
(12) Patent Application Publication
(10) Pub. No.: US 2009/0198244 A1
(43) Pub. Date: Aug. 6, 2009

(54) Instrument for Implanting a Wrist Prosthesis

(76) Inventor: David A. Leibel, Princeton, MN (US)

Correspondence Address: MORGAN, LEWIS & BOCKIUS LLP, 1701 MARKET STREET, PHILADELPHIA, PA, 19103-2921, US

(21) Appl. No.: 12/085,119

(22) PCT Filed: Nov. 20, 2006
(86) PCT No.: PCT/US06/44963

§ 371 Date: Jan. 12, 2009

Related U.S. Application Data
(60) Provisional application No. 60/738,276, filed on Nov. 18, 2005.

(51) Int. Cl. A61B 17/58; A61F 5/00

(52) U.S. Cl. 606/99; 606/87

(57) ABSTRACT

A wrist instrument and methods for identifying a location for implantation of a wrist prosthesis are disclosed in the present application.

### BACKGROUND OF THE INVENTION

The wrist joint is located at the intersection of the radius and ulna with the carpals and metacarpals. When the wrist joint is damaged due to, for example, fracture or arthritis, it is common to replace the wrist joint with a wrist prosthesis. Disclosed herein are instruments and methods used in identifying a location for implanting a wrist prosthesis.

### SUMMARY OF THE INVENTION

The present invention relates to instruments and methods used in implantation of a wrist prosthesis between the carpal bones, for example, the lunate fossa, and the radius. In one embodiment, the instrument disclosed in the present invention comprises a radial block component. In an embodiment of the invention, the radial block includes a first angled surface configured to align with a second angled surface of the radius bone, or the radial styloid, located on the radial side of the radius bone. In an embodiment of the invention, the first angled surface includes an angle of from about forty to about fifty degrees relative to the dorsal surface of the radial block.

In another embodiment of the present invention, the radial block is configured to accommodate additional instruments for implanting the wrist prosthesis. Such additional instruments include, but are not limited to a carpal cutting guide, a radial milling cutter, and a radial broaching guide pin.

In another embodiment, the radial block has at least one opening through the dorsal-palmar axis of the block. In an embodiment of the invention, the opening is configured to accommodate a fastener. In another embodiment, the fastener includes a k-wire, a pin, a rod, a staple, or a screw.

In another embodiment of the present invention, a spacer is included in the instrument of the present invention. In one embodiment, the spacer includes a tab portion. In another embodiment, the spacer includes a spacer body. In an embodiment of the invention, the spacer body abuts the radial block. In another embodiment, the tab aligns with the carpal bone, for example, the lunate fossa.

In another embodiment of the present invention, the instrument also includes a handle and a set screw. In an embodiment of the invention, the handle is configured to accommodate the set screw and the set screw is configured to connect the handle and the radial block.

In another embodiment of the present invention, the instrument also includes a carpal cutting guide. In one embodiment, the carpal cutting guide includes a proximal end, a distal end, and a carpal cutting jig. In another embodiment, the proximal end of the carpal cutting guide is accommodated by the spacer and the carpal cutting jig abuts the spacer. In another embodiment of the present invention, the distal end of the carpal cutting guide aligns with a third metacarpal bone.

The present invention also includes a method for implanting a wrist prosthesis between a radius bone and a carpal bone. In an embodiment of the invention, the method includes placing a radial block on the dorsal side of the radius bone, such that a first angled surface of the radial block abuts a second angled surface on the radial side of the radius bone. In an embodiment of the invention, the first angled surface of the radial block includes an angle of from about forty to about fifty degrees relative to a dorsal surface of the radial block.

In another embodiment, the method also includes placing a spacer having a tab portion and a spacer body against the distal surface of the radial block, such that the tab portion is oriented between the radius and the carpal bones, and aligns with at least one carpal bone, for example, the lunate fossa, and the spacer body abuts the radial block.

In an embodiment of the invention, the method also includes securing the radial block to the radius bone with a fastener selected from the group consisting of a k-wire, a screw, a pin, a rod, and a staple.

In an embodiment of the invention, the method also includes cutting the carpal bone with a carpal cutting guide.

In another embodiment of the present invention, the method includes milling the radius with a radial milling cutter.

In another embodiment of the present invention, the method includes broaching the radius bone with the radial broaching guide pin.

In another embodiment of the present invention, the method includes inserting a radial portion of a wrist implant into the radius bone.

In an embodiment of the invention, the radial block establishes the radial-ulnar location of the wrist prosthesis. In another embodiment of the invention, the radial block, through placement of a spacer between the radial and carpal bones, establishes the proximal-distal location of the wrist prosthesis.

### BRIEF DESCRIPTION OF THE DRAWINGS

The foregoing summary, as well as the following detailed description of various embodiments of the invention, will be better understood when read in conjunction with the appended drawings. Drawings are provided for the purpose of illustrating certain embodiments of the invention. It should be understood, however, that the invention is not limited to the precise arrangements and instrumentalities shown, including the recited dimensions. In the drawings:

FIG. 1A depicts components of a wrist instrument according to the present invention. FIG. 1B depicts an embodiment of an assembled wrist instrument according to the present invention.

FIG. 2 depicts a top view of the placement of a wrist instrument according to the present invention against the radius and carpal bones.

FIG. 3 depicts a side view of the placement of a radial block according to the present invention against the radius bone.

FIG. 4 depicts a top view of the placement of a radial block according to the present invention against the radius bone.

FIG. 5 depicts a front view of the placement of a radial block according to the present invention against the radius bone.

FIG. 6 depicts an isometric view of a wrist instrument according to the present invention. FIG. 6 also depicts fasteners being inserted into a radial block according to the present invention and through the radius bone to secure the radial block to the radius bone.

FIG. 7 depicts an isometric view of an assembled and placed wrist instrument according to the present invention. FIG. 7 also depicts a carpal saw being placed into a carpal cutting jig portion of an instrument according to the present invention, and the carpals being cut along the jig axis.

FIG. 8A depicts an isometric view of an assembled and placed wrist instrument according to the present invention. Also shown is a step in a method of implanting a wrist prosthesis according to the present invention. A milling cutter is attached to a radial block according to the present invention, and cortical bone is removed. FIG. 8B depicts an isometric view of a portion of a milling cutter according to the present invention.

FIG. 9 depicts an isometric view of an assembled and placed wrist instrument according to the present invention. Also shown is a step in a method of implanting a wrist prosthesis according to the present invention. A radial broaching guide pin is attached to a radial block according to the present invention, and a k-wire is drilled centrally into the radius where a wrist implant will be positioned.

FIG. 10 depicts an isometric view of an assembled and placed wrist instrument according to the present invention. Also shown in a step in a method of implanting a wrist prosthesis according to the present invention. A cannulated broach slides over a k-wire guide, to file out the distal radius into the shape of the implant.

FIG. 11 depicts an isometric view of an assembled and placed wrist instrument according to the present invention. Also shown in a step in a method of implanting a wrist prosthesis according to the present invention. As shown, a carpal guide plate is placed over a carpal surface, and held in place with a k-wire.

FIG. 12 illustrates a k-wire used to hold a carpal guide plate in place being cut down as far as possible, in accordance with the present invention.

FIGS. 13A and 13B illustrate a carpal ball of a wrist prosthesis being measured for size on a carpal guide plate of a wrist prosthesis, in accordance with the present invention.

FIG. 14 illustrates drilling of holes into the carpal bones for securing the carpal plate of a wrist prosthesis to the carpals, in accordance with the present invention.

FIG. 15 illustrates broaching the carpal bones for positioning the carpal implant of a wrist prosthesis, in accordance with the present invention.

FIG. 16 illustrates installing the carpal plate, fasteners, and carpal ball of a wrist prosthesis, in accordance with the present invention.

### DETAILED DESCRIPTION

The present invention relates to instruments and methods for use in implanting a medical device, (e.g., a wrist prosthesis). In one embodiment, the medical device (e.g., a wrist prosthesis) generally will have at least a radial portion and a carpal portion.

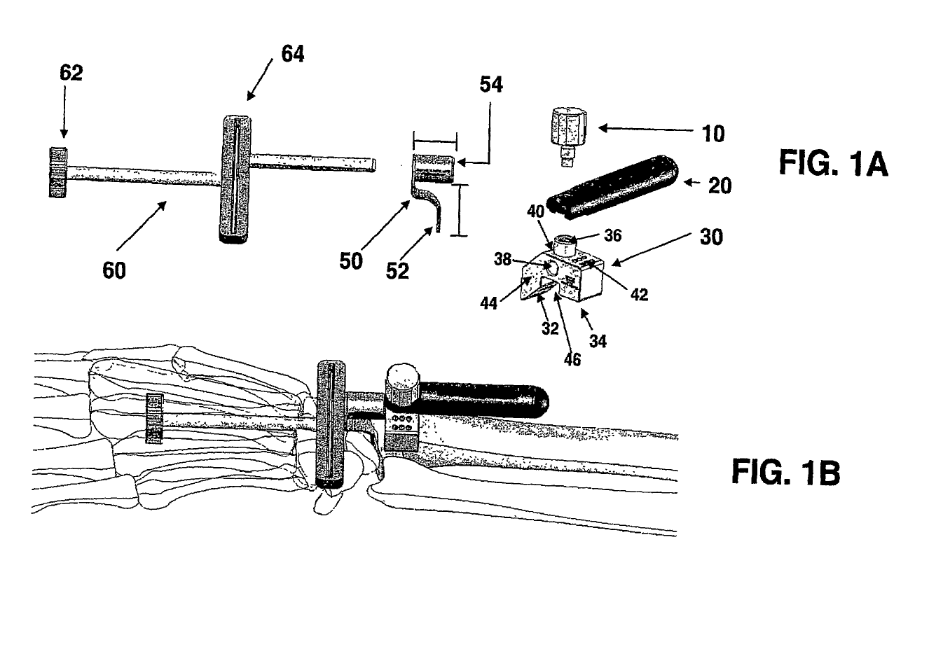

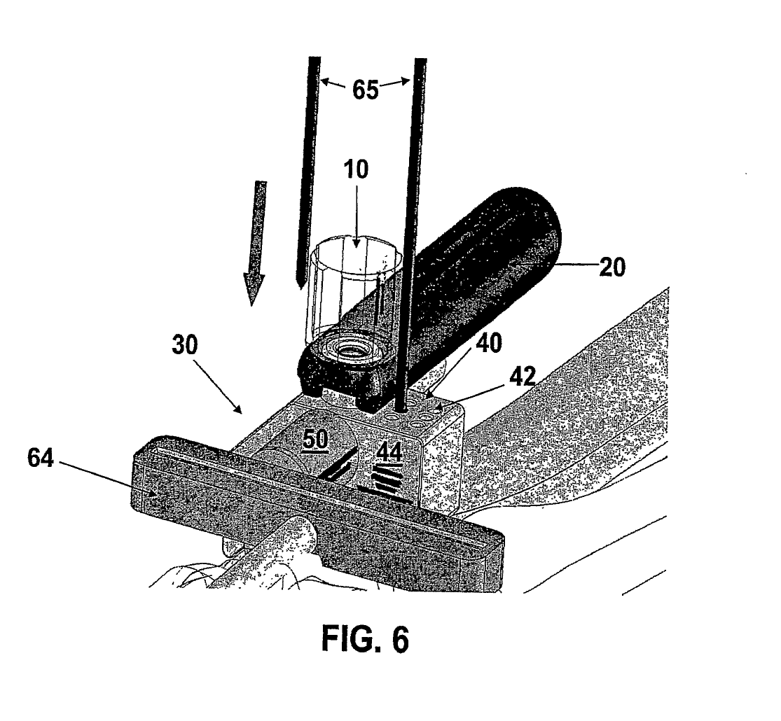

Referring to FIGS. 1A and 6, in an embodiment of the invention, the instrument of the present invention includes a radial block 30, a spacer 50, a handle 20, and a set screw 10. In one embodiment, radial block 30 includes an angled surface 32, a distal surface 44, a dorsal surface 40, a palmar surface 34, an instrument attachment aperture 38 configured to accommodate additional instruments, at least one opening 42 on dorsal surface 40 that extends from dorsal surface 40 through palmar surface 34, to accommodate one or more fasteners 65 (e.g., k-wires, pins, screws, rods, posts, staples, or other fasteners), a notch 46, and a handle attachment 36.

In one embodiment, angled surface 32 is angled at from about 30 to about 60 degrees relative to dorsal surface 40 of radial block 30. In another embodiment, angled surface 32 is angled at from about 40 to about 50 degrees. In another embodiment, angled surface 32 is angled at about 45 degrees. In another embodiment of the invention, angled surface 32 includes at least one opening 42 that extends through radial block 30 to dorsal surface 40 of radial block 30.

In an embodiment of the invention, notch 46 is configured to fit over or straddle Lister's Tubercle so to minimize the number of cuts necessary to implant a wrist prosthesis according to the present invention.

In an embodiment of the invention, handle 20 includes a metal rod, which serves as a radiographic marker when viewing placement of the instrument via fluoroscopy, x-ray, or another internal viewing method. In an embodiment of the invention, a surgeon views the position of the metal rod in handle 20 to confirm that radial block 30 is perpendicular to the long axis of the radius bone.

In an embodiment of the invention, referring to FIG. 1A, spacer 50 includes a spacer tab 52 and a spacer body 54. Spacer tab 52 is configured to align with a carpal bone. In an embodiment of the present invention, spacer tab 52 is configured to align with the lunate fossa. In another embodiment, spacer body 54 is configured to abut against radial block 30. More specifically, the proximal end of spacer body 54 abuts against distal surface 44 of radial block 30.

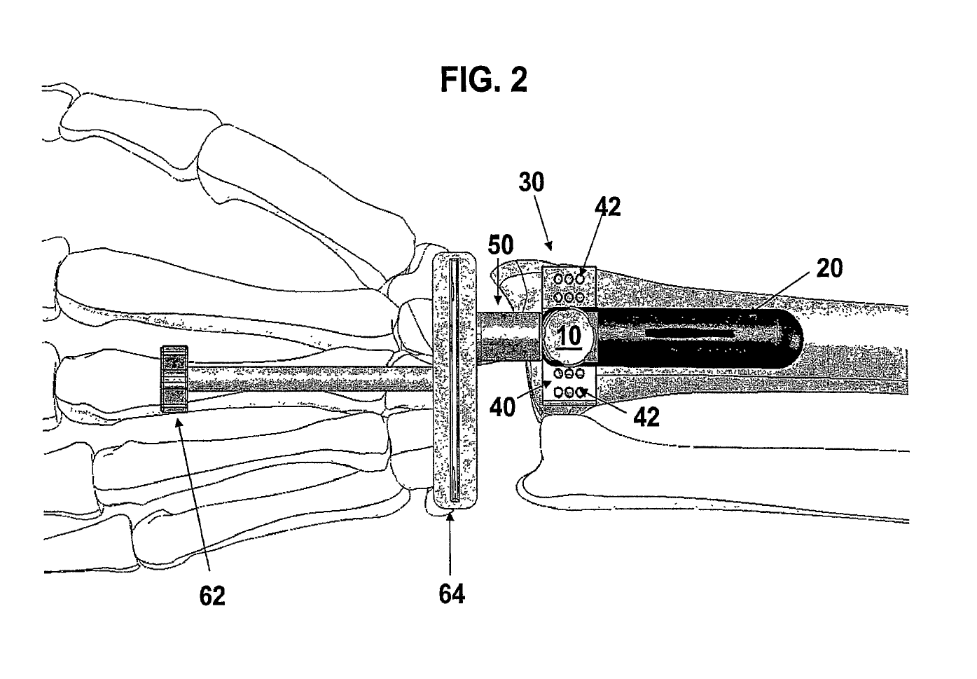

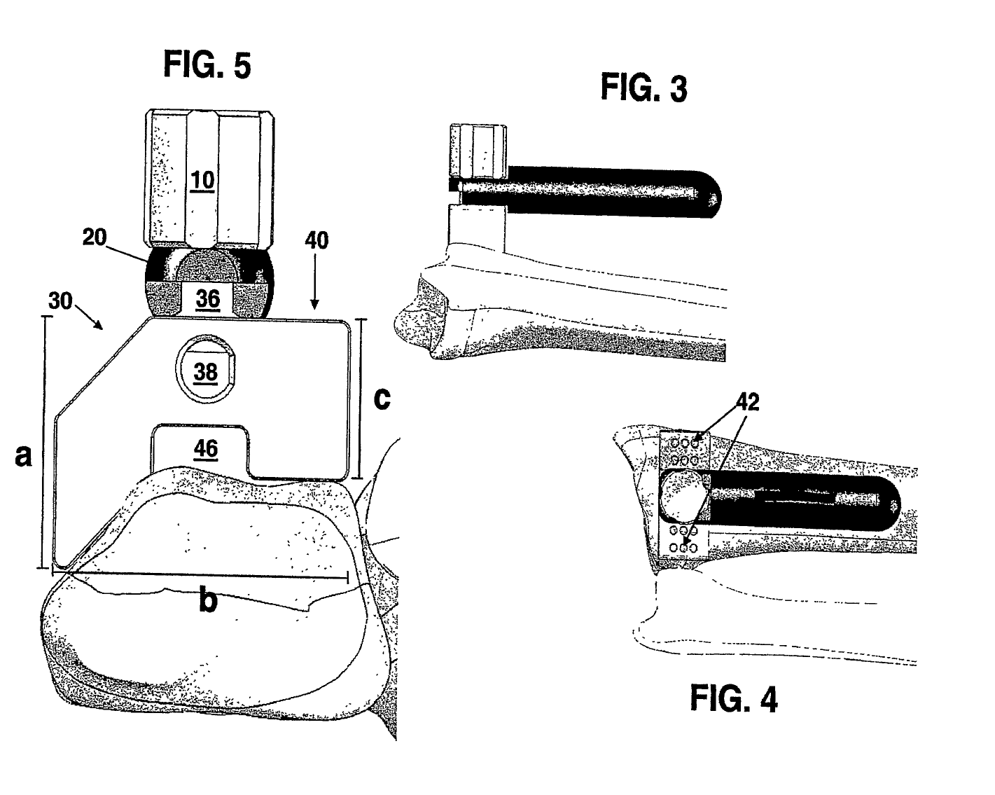

Referring to FIG. 2, a top view of an assembled instrument according to the present invention is shown. Distal end 62 of carpal cutting guide 60 aligns with the third metacarpal. Carpal cutting jig 64 abuts against spacer 50, and the proximal end of spacer 50 abuts against radial block 30. FIGS. 3-5 illustrate side, top, and front views, respectively, of an assembled instrument according to the present invention without cutting guide 60 attached.

Referring to FIG. 5, in an embodiment of the present invention, with reference to placement of radial block 30 with respect to the radius bone, the dimensions of radial block 30 are from about 0.5 to about 2.5 inches in the radial-ulnar direction (dimension b), from about 0.25 to about 1 inch in the proximal-distal direction (dimension not shown), from about 0.5 to about 1.5 inches in the dorsal-palmar direction on the radial side (dimension a), and from about 0.25 to about 1 inch in the dorsal-palmar direction on the ulnar side (dimension c). In another embodiment, the dimensions of radial block 30 are about 1.25 inches in the radial-ulnar direction, about 0.5 inch in the proximal-distal direction, about 1 inch in the dorsal-palmar direction on the radial side, and about 0.5 inch in the dorsal-palmar direction on the ulnar side.

In an embodiment of the invention, one of the benefits of radial block 30 according to the present invention is that it has an instrument attachment aperture 38 to accommodate additional instrumentation necessary for implanting a medical device, such as a wrist prosthesis. In an embodiment of the invention, the additional instrumentation are connected to radial block 30 to ensure precision in implanting the medical device (e.g., a wrist prosthesis).

In an embodiment of the invention, spacer 50 is available in a variety of sizes, including, but not limited to, extra small, small, medium, large, and extra large. In an embodiment of the invention, spacer 50 has a diameter of from about 0.125 to about 1 inch. In another embodiment, spacer 50 has a diameter of from about 0.25 to about 0.875 inch. In another embodiment, spacer 50 has a diameter of about 0.375 inch. In an embodiment of the invention, the length of spacer 50 ranges from about 0.250 to about 1 inch. In another embodiment, the length of spacer 50 ranges from about 0.3 to about 0.875 inch. In yet another embodiment, the length of spacer 50 ranges from about 0.375 to about 0.535 inch. In an embodiment of the invention, the length of spacer 50 is 0.375 inch. In another embodiment, the length of spacer 50 is 0.455 inch. In another embodiment, the length of spacer 50 is 0.535 inch. The size of spacer 50 used in the method for implanting a wrist prosthesis will depend upon the size of the wrist being implanted, as discussed more fully below.

In an embodiment of the invention, spacer 50 includes a tab portion 52. Tab portion 52 extends in length from about 0.5 to about 1.75 inches. In another embodiment, tab portion 52 extends in length from about 0.75 to about 1.5 inches. In another embodiment, tab portion 52, extends in length from about 1 to about 1.5 inches. In another embodiment, tab portion 52 is about 1 inch in length. In another embodiment, tab portion 52 is about 1.5 inches in length.

In an embodiment of the invention, referring to FIG. 1A, the instrument includes radial block 30, a handle 20, and a set screw 10. In one embodiment, the instrument includes radial block 30, handle 20, set screw 10, and a carpal cutting guide 60. In an embodiment of the invention, carpal cutting guide 60 includes a distal end 62, a proximal end 66, and a carpal cutting jig 64.

In an embodiment of the invention, the instrument is assembled and placed as follows. Set screw 10 is inserted through handle 20 into handle attachment 36 of radial block 30. Radial block 30 with handle 20 and set screw 10 attached, is placed on the dorsal surface of the radius bone and oriented such that angled surface 32 contacts the angled portion of the radius bone on the radial side (i.e., the radial styloid), as shown in FIG. 5. Palmar surface 34 of radial block 30 contacts the dorsal surface of the radius bone, and distal surface 44 of radial block 30 faces the hand, as shown in FIGS. 3-5.

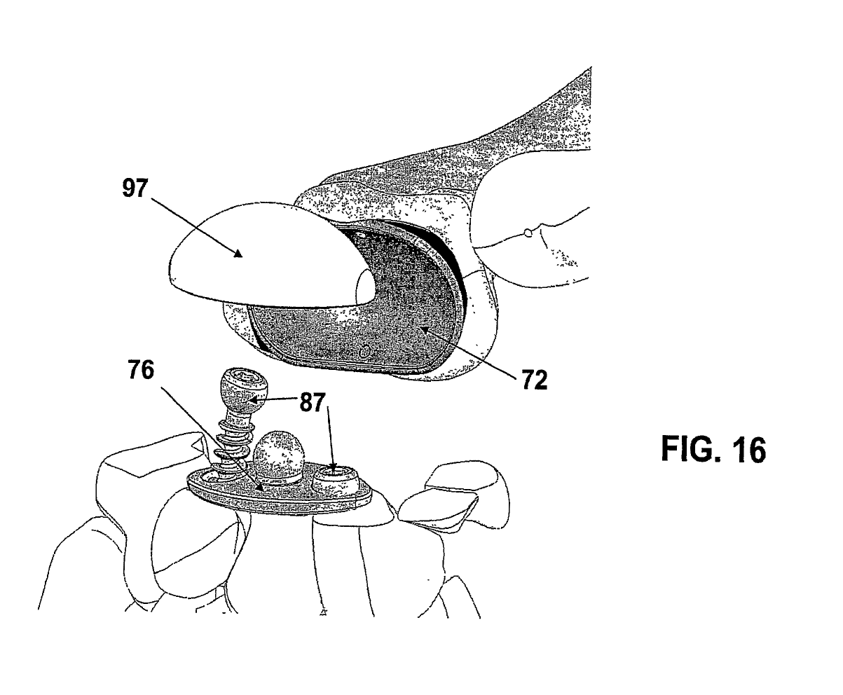

When used with a wrist prosthesis, such as the wrist prosthesis shown in FIG. 16 and described in U.S. patent application Ser. No. 10/897,317, filed on Jul. 22, 2004, spacer 50 is placed up against distal surface 44 of radial block 30 and proximal end 66 of carpal cutting guide 60 is inserted into spacer 50 and adjusted such that carpal cutting jig 64 abuts spacer 50. Distal end 62 of carpal cutting guide 60 aligns with the dorsal surface of the third metacarpal. One embodiment of the assembled instrumentation is shown in FIG. 5.

In an embodiment of the invention, the instrumentation is fabricated from biocompatible metal, such as stainless steel, cobalt chrome, or titanium. In another embodiment, the instrumentation is fabricated from a rigid plastic or polymer, such as, for example, polyurethane, polyethylene, or polypropylene. In another embodiment of the invention, the instrumentation of the present invention may be injection-molded, casted, or machine molded.

A method for identifying an optimal location for implantation of a wrist prosthesis is also disclosed in the present application. In an embodiment of the invention, the method includes fitting radial block 30 on the dorsal surface of a radius bone and fitting spacer 50 between radial block 30 and carpal cutting guide 60, as illustrated, for example, in FIG. 2.

In an embodiment of the present invention, radial block 30 establishes the radial-ulnar location of a wrist prosthesis, and spacer 50 establishes the proximal-distal location of radial block 30, which, in turn, establishes the proximal-distal location of the wrist prosthesis. In an embodiment of the invention, the precision with which the prosthesis location is determined is attributed, in part, to angled surface 32 of radial block 30. Since the radius bone naturally has about a 45 degree angle on the dorso-radial side of the radius bone, this surface acts as a marker for radial block 30 to contact. Thus, radial block 30 of the present invention approximates the angled surface of the radius bone and rests against this surface to ensure maximum placement precision of the wrist prosthesis in the radial-ulnar direction, and ultimately, the proximal-distal direction of the wrist prosthesis.

In an embodiment of the present invention, the method includes aligning handle 20 along the length of a radius bone, aligning radial block 30 on the radius bone, spacer 50, and carpal cutting guide 60 as discussed above and as shown in FIGS. 3-5. In an embodiment of the invention, notch 46 straddles Lister's Tubercle. In an embodiment of the invention, the positioning of these components is verified using fluoroscopy, x-rays, or another method for viewing internal structures known in the art. As discussed above, handle 20 includes a metal rod for visualization using fluoroscopy, x-rays, or another method for viewing internal structures. In one embodiment, radial block 30 is secured to the radius bone by at least one fastener 65, for example, k-wires, inserted through openings 42, as shown in FIG. 6. In another embodiment other fasteners useful in the present invention include pins, rods, posts, staples, and screws.

In another embodiment, the method also includes aligning spacer 50 and carpal cutting guide 60 as discussed above and as shown in FIGS. 1B and 2. In one embodiment, a surgeon performing this surgery would first determine the size of the spacer necessary for the surgery. In an embodiment of the invention, the spacer serves to maintain a particular distance between the carpals and the radius bone, and the size of the spacer will depend on the size of the wrist being implanted. This is usually determined by x-ray examination; however any standard method can be used in accordance with the present invention.

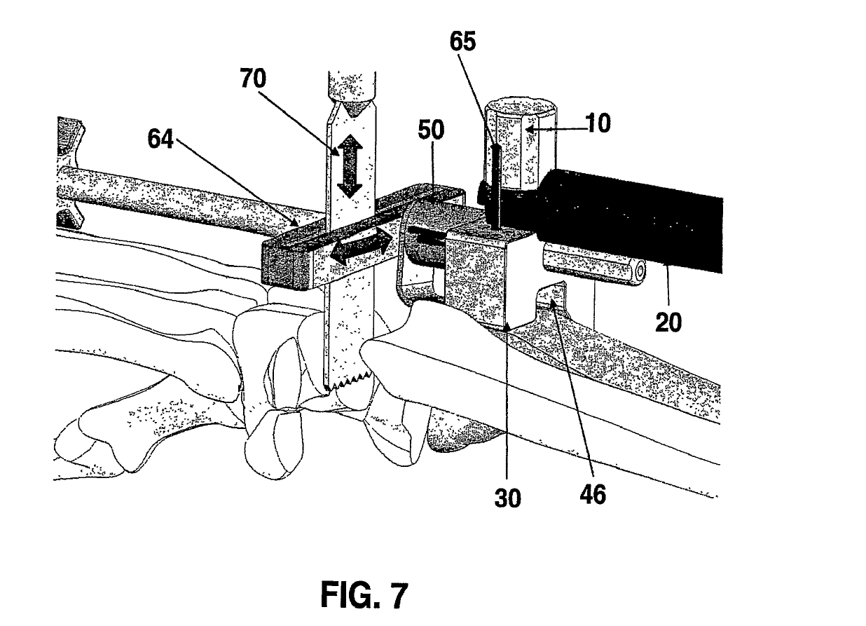

In an embodiment of the invention, after radius block 30, spacer 50 and carpal cutting guide 60 are in place and secured with fastener 65, a carpal saw 70 is inserted into carpal cutting jig 64 and the carpals are cut along the carpal cutting jig 64 axis, as shown in FIG. 7. The arrows on carpal saw 70 indicate the cutting direction of carpal saw 70. The arrows on carpal cutting jig 64 indicate the direction of carpal cutting jig 64 axis.

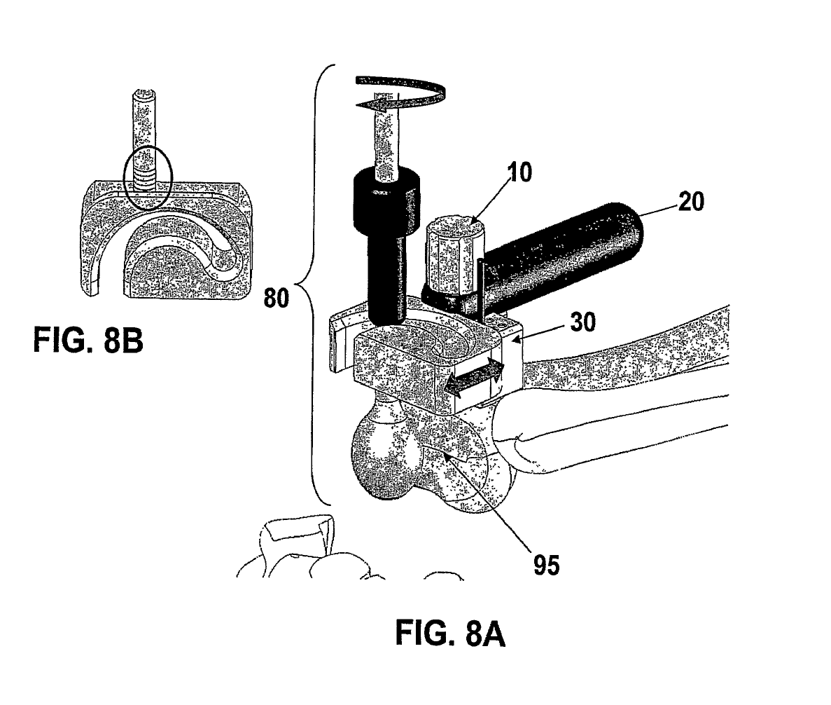

In another embodiment of the invention, a radial milling guide 80 is mounted onto radial block 30 via instrument attachment aperture 38, and cortical bone is removed from the radius. In one embodiment, radial milling guide 80 removes the cortical bone in such a manner as to leave a concave surface 95 on the radius bone, as shown in FIG. 8A. In an embodiment of the invention, radial milling guide 80 is adjusted in the proximal-distal direction (as indicated by the arrows in FIG. 8A) so that the distal radius can be cut gradually in a number of small steps.

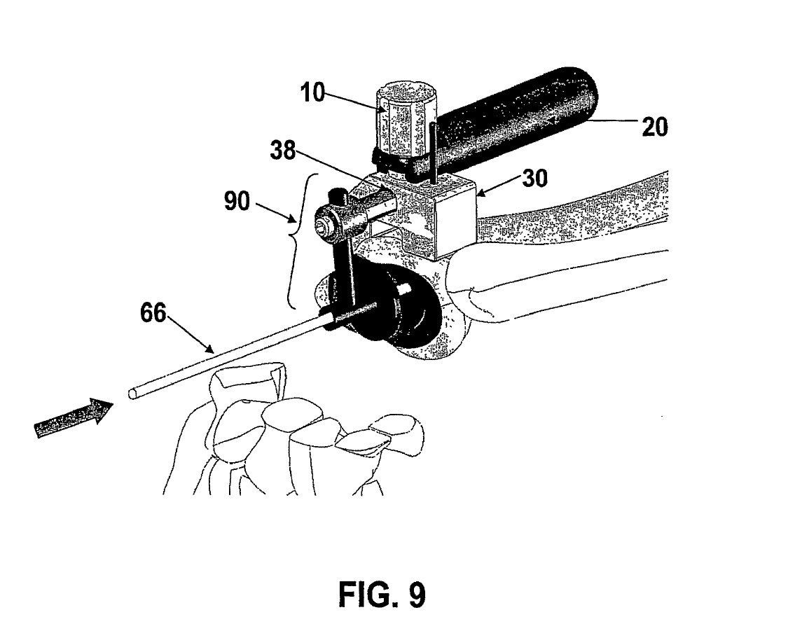

In another embodiment of the invention, a radial broaching guide pin 90 is mounted onto radial block 30 via instrument attachment aperture 38, and the central radius is broached where the radial portion of the wrist prosthesis is to be placed. In an embodiment of the invention, the central radius is broached by drilling a k-wire 66, for example, a 2 millimeter k-wire, through radial broaching guide pin 90, as shown in FIG. 9.

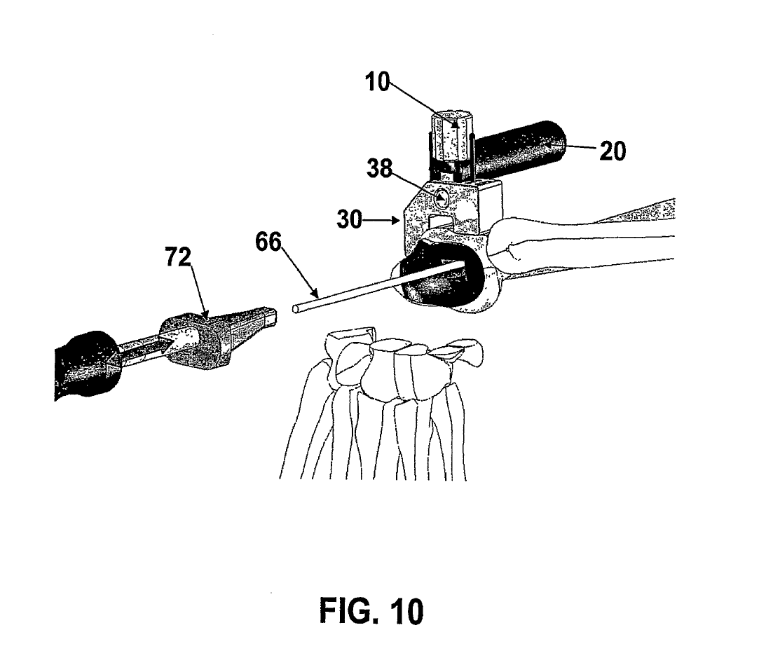

In an embodiment of the invention, a hole is created (e.g., by broaching) in the center of the distal radius for accepting the radial portion 72 of a wrist prosthesis. In an embodiment of the present invention, a cannulated broach is inserted over k-wire 66 as a guide to create the hole in the distal radius to accept radial portion 72 of the wrist prosthesis, as shown in FIG. 10.

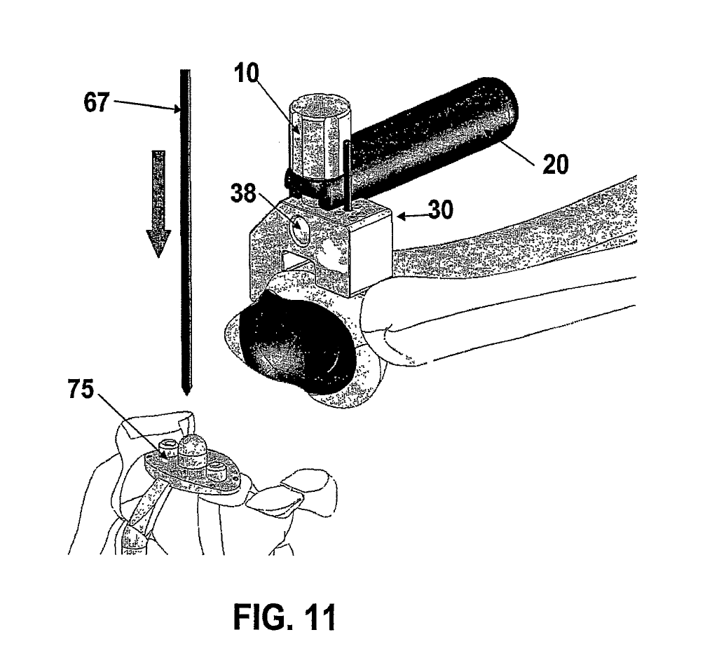

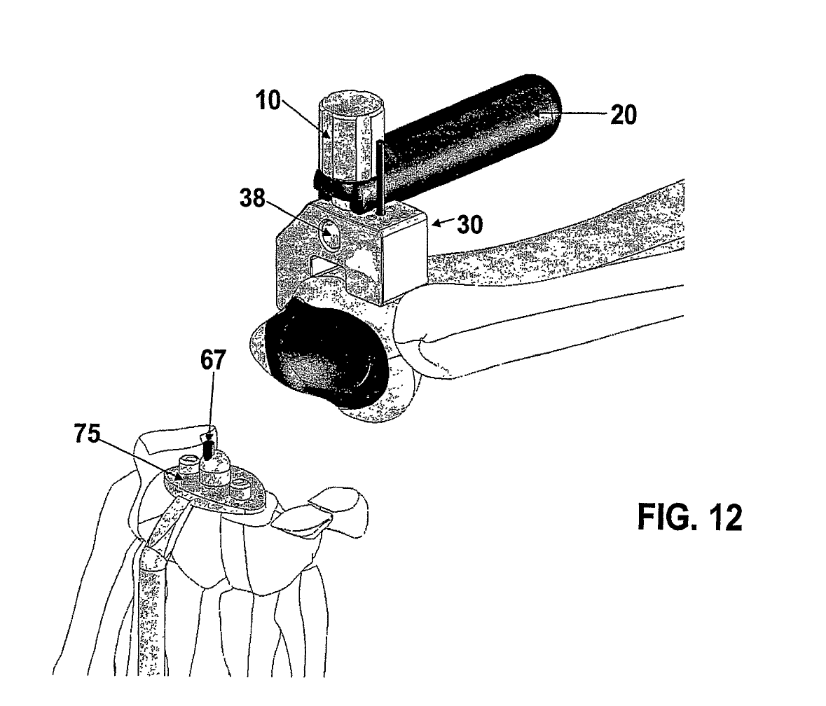

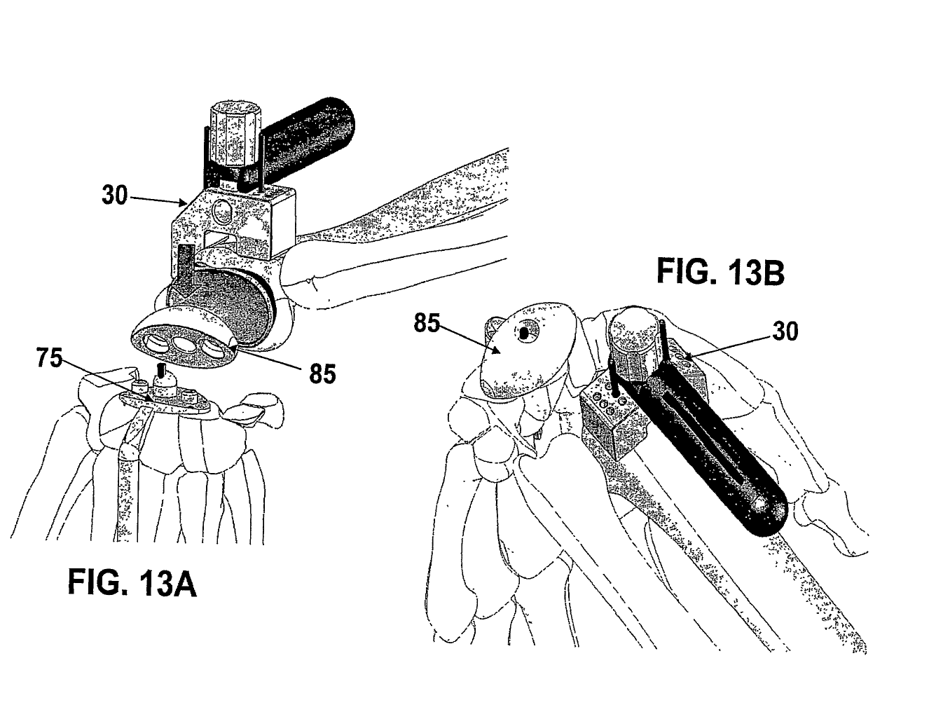

In an embodiment of the present invention, a carpal guide plate 75 is fixed with a fastener over the cut surface of the carpal bones, for example, over the capitate, and the third metacarpal. In one embodiment, a k-wire 67 is drilled into the center of carpal guide plate 75 to mark the location and appropriate depth for the carpal portion 76 of a wrist prosthesis, as shown in FIG. 11. In one embodiment, fluoroscopy, x-rays, or another method for viewing internal structures known in the art, is used to assess the correct depth and position of k-wire 67 as it is inserted into carpal guide plate 75. In another embodiment, k-wire 67 is cut down as far as possible to carpal guide plate 75, as shown in FIG. 12. In yet another embodiment, a carpal ball guide 85 is fitted over carpal guide plate 75 to ensure correct size of the carpal ball portion 97 of the wrist prosthesis, as shown in FIGS. 13A and 13B.

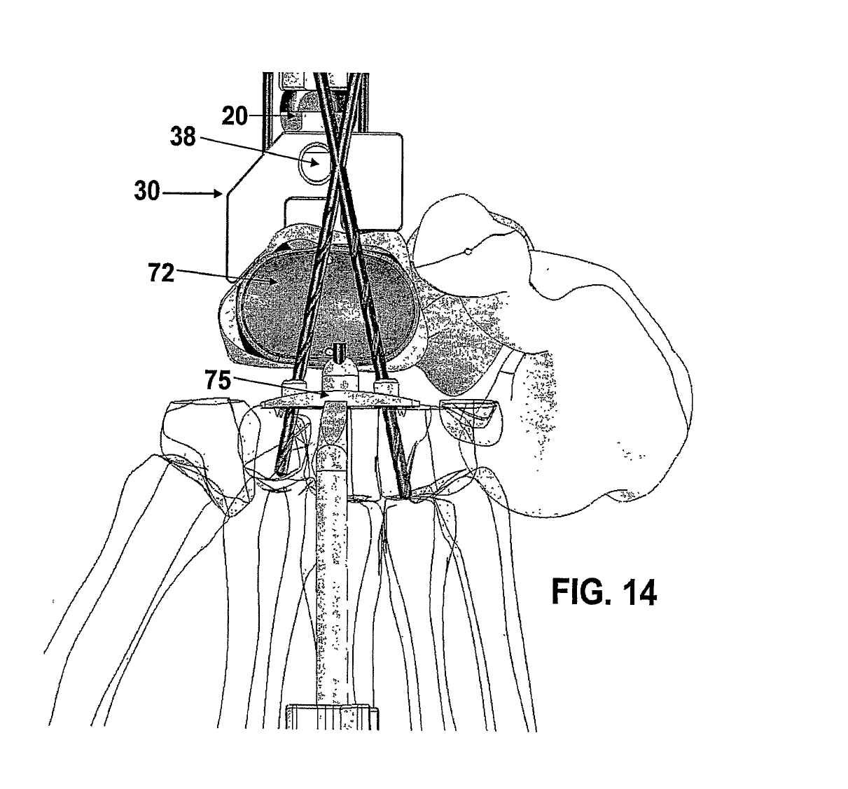

In an embodiment of the present invention, carpal holes are drilled where one or more fasteners will be inserted to hold carpal portion 76 of the wrist prosthesis in place. In an embodiment of the invention, holes are drilled on either side of the midpoint of carpal guide plate 75 to an appropriate depth, as shown in FIG. 14. In one embodiment, the carpal holes are drilled to the same depth. In another embodiment, the carpal holes are drilled to different depths. In yet another embodiment, the carpal holes are drilled at an angle to carpal guide plate 75. In yet another embodiment, the carpal holes are drilled perpendicular to carpal guide plate 75.

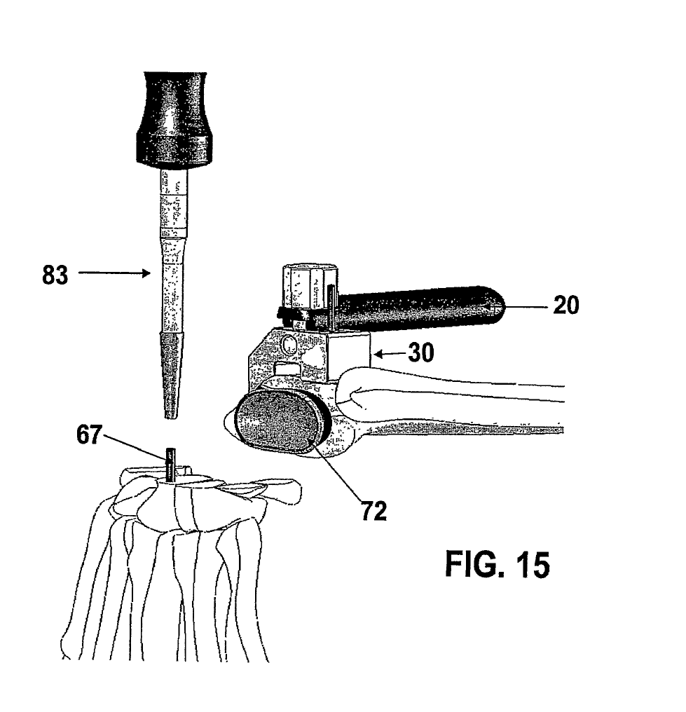

In an embodiment of the present invention, carpal guide plate 75 is removed and, using k-wire 67 as a location guide, the carpal bones are broached for positioning of carpal portion 76 of the wrist prosthesis using a broaching instrument 83, as shown in FIG. 15.

In an embodiment of the invention, carpal portion 76 of the wrist prosthesis is inserted into the carpal bones and secured with additional fasteners, such as screws, k-wires, posts, rods, staples, or pins. In an embodiment of the invention, carpal portion 76 of the wrist prosthesis is secured with at least one screw 87, as shown in FIG. 16. In another embodiment, carpal ball 97 fits over the top of carpal portion 76 of the wrist prosthesis, as shown in FIG. 16.

It will be apparent to those skilled in the art that various modifications and variations can be made in the device of the present invention without departing from the scope of the invention. Thus, it is intended that the present invention cover the modifications and variations of the present invention. Any and all publications, patents, and patent applications are herein incorporated by reference in their entirety.

What is claimed is:

1. An instrument for implanting a wrist prosthesis comprising:
a. a radial block, including a first angled surface configured to align with a second angled surface of a radius bone; and
b. a spacer configured to align with a carpal bone and configured to abut the radial block.

2. The instrument of claim 1, wherein the spacer comprises a tab and a spacer body.

3. The instrument of claim 2, wherein the tab is configured to align with the carpal bone.

4. The instrument of claim 1, wherein the carpal bone is a lunate fossa bone.

5. The instrument of claim 1, wherein the first angled surface comprises an angle of from about forty to about fifty degrees relative to a dorsal surface of the radial block.

6. The instrument of claim 1, further comprising a handle and a set screw.

7. The instrument of claim 1, wherein the radial block component further comprises at least one opening through a dorsal-palmar axis.

8. The instrument of claim 7, wherein the at least one opening accommodates a fastener.

9. The instrument of claim 8, wherein the fastener is selected from the group consisting of a k-wire, a screw, a pin, a rod, a post, and a staple.

10. The instrument of claim 1, wherein the radial block is configured to accommodate additional instruments.

11. The instrument of claim 10, wherein the additional instruments are selected from the group consisting of a carpal cutting guide, a radial milling cutter, and a radial broaching guide pin.

12. The instrument of claim 1, wherein the angled surface of the radius bone is located on a radial side of the radius bone.

13. An instrument for implanting a wrist prosthesis comprising:
a. a radial block comprising a first angled surface configured to align with a second angled surface of a radius bone, wherein the first angled surface comprises an angle of from about forty to about fifty degrees relative to a dorsal surface of the radial block;
b. a spacer comprising a spacer body and a tab, wherein the tab is configured to align with a lunate fossa bone, and wherein the spacer body is configured to abut the radial block;
c. a handle and a set screw, wherein the handle is configured to accommodate the set screw and the set screw is configured to connect the handle and the radial block; and
d. a carpal cutting guide comprising a proximal end, a distal end, and a carpal cutting jig, wherein the proximal end is accommodated by the spacer and the carpal cutting jig abuts the spacer, and wherein the distal end aligns with a third metacarpal bone.

14. A method for implanting a wrist prosthesis between a radius bone and a carpal bone, the method comprising:
a. placing a radial block comprising a first angled surface on a dorsal side of the radius bone, such that the first angled surface abuts a second angled surface on a radial side of the radius bone; and
b. placing a spacer against the radial block, such that the spacer is oriented between the radius and the carpal bone and aligns with the carpal bone.

15. The method of claim 14, further comprising securing the radial block to the radius bone with a fastener selected from the group consisting of a k-wire, a screw, a pin, a rod, a post, and a staple.

16. The method of claim 14, further comprising attaching a carpal cutting guide to the radial block and cutting the carpal bone with the carpal cutting guide.

17. The method of claim 14, further comprising attaching a radial milling cutter to the radial block and milling the radius with the radial milling cutter.

18. The method of claim 14, further comprising attaching a radial broaching guide pin to the radial block and broaching the radius bone with the radial broaching guide pin.

19. The method of claim 14, further comprising inserting a radial portion of the wrist implant into the radius bone.

20. The method of claim 14, wherein the carpal bone is the lunate fossa bone.

21. The method of claim 14, wherein the spacer comprises a tab that aligns with the carpal bone and a spacer body that abuts the radial block.

22. The method of claim 14, wherein the first angled surface comprises an angle of from about forty to about fifty degrees relative to a dorsal surface of the radial block.
